(19) United States
(12) Patent Application Publication
(10) Pub. No.: US 2005/0065587 A1
(43) Pub. Date: Mar. 24, 2005

(54) Implantable lead with magnetic jacket

(76) Inventor: Mark Gryzwa, Woodbury, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/670,156

(22) Filed: Sep. 24, 2003

(51) Int. Cl. A61N 1/05

(52) U.S. Cl. 607/122

(57) ABSTRACT

An implantable lead assembly includes a lead body extending from a proximal end to a distal, where the lead body has an intermediate portion therebetween. The lead body includes an insulating layer and a conductor disposed within the insulating layer. The insulating layer surrounds the conductor. An electrode is coupled to the lead body, and the electrode is in electrical communication with the conductor. At least one magnetic jacket is disposed within the insulating layer, and the at least one magnetic jacket surrounds the conductor.

### TECHNICAL FIELD

The present invention relates generally to implantable lead assemblies. More particularly, it pertains to implantable lead assemblies having magnetic jackets for the reduction of electromagnetic interference in implantable medical devices.

### BACKGROUND

Increased use of electromagnetic interference (EMI) sources, such as cellular phones, keyless entry systems and electronic anti-theft systems has provided an increased demand for implantable medical devices that are less susceptible to such interference. In implantable medical devices, for example, implantable pacemakers, the leads used unintentionally act as antenna and tend to collect stray electromagnetic signals which are then transmitted to the pacemaker. This ‘antenna effect,’ in some instances causes incorrect interpretation of electromagnetic interference as cardiac activity by the pacemaker. Such misinterpretation by the pacemaker can result in an inability to sense actual cardiac activity and therefore prevent needed pacing therapy. In some circumstances misinterpretation of electromagnetic interference as cardiac activity can cause undesirable pacing.

In an attempt to address electromagnetic interference, feedthrough filter capacitors have been developed. One example of a feedthrough filter capacitor is described in U.S. Pat. No. 5,333,095. The feedthrough capacitor described therein has a ceramic capacitor that is coupled to a terminal pin. The capacitor is designed to decouple unwanted electromagnetic interference picked up by the antenna effect of the leads before such interference can interact with the pacemaker. One disadvantage of feedthrough capacitors is the need for multiple components, which are expensive to manufacture and assemble. Additionally, feedthrough capacitors increase the volume of a pacemaker.

Additionally, shunts have been developed to divert electromagnetic interference away from the sensitive electronics of implantable medical devices. One example of such a device is described in U.S. Pat. No. 5,683,434. Installation of the shunt requires soldering or other similar means of coupling the shunt to the pacemaker. Additionally, to operate correctly the shunt requires a thin dielectric layer to separate it from the pacemaker housing. Provision of such a dielectric layer requires another manufacturing step. Further, the shunt also increases the volume of a pacemaker.

What is needed is an implantable lead assembly that overcomes the shortcomings of previous implantable lead assemblies. What is further needed is an implantable lead assembly that decreases the susceptibility of implantable medical devices to electromagnetic interference.

### SUMMARY

An implantable lead assembly includes a lead body which extends from a proximal end to a distal end, the lead body has an intermediate portion therebetween and an insulating layer. A conductor is disposed within the insulating layer and the insulating layer surrounds the conductor. An electrode is coupled to the lead body and in electrical communication with the conductor. A magnetic jacket is disposed within the insulating layer, and the magnetic jacket surrounds the conductor.

Several options for the implantable lead assembly follow. In one option, the magnetic jacket is disposed adjacent to the conductor. In another option, the magnetic jacket is electrically non-conductive. In yet another option, the magnetic jacket includes a substrate and magnetic particles disposed therein. In a further option, the magnetic jacket includes interstitial magnetic particles that are disposed substantially throughout the insulating layer. A second conductor is disposed within the insulating layer, and the insulating layer surrounds the second conductor, in one option.

In another embodiment a method comprises increasing the inductance of an implantable lead assembly with the inductance of a magnetic jacket. The implantable lead assembly includes a lead body extending from a proximal end to a distal end, and the lead body includes an insulating layer and a conductor disposed within the insulating layer. An electrode is coupled to the lead body and in electrical communication with the conductor. The insulating layer surrounds the conductor. The magnetic jacket is disposed within the insulating layer and surrounds the conductor. The method further includes exposing a pulse generator and the implantable lead assembly coupled thereto to electromagnetic interference. Additionally, the method includes isolating the pulse generator and the implantable lead assembly from electromagnetic interference.

Several options for the method follow. In one option, for example, increasing the inductance of the implantable lead assembly includes adding the inductance of the magnetic jacket with an inductance of the conductor. In another option, isolating the pulse generator and the implantable lead assembly from electromagnetic interference includes decreasing the antenna efficiency of the implantable lead assembly. In yet another option, isolating the pulse generator and the implantable lead assembly includes decreasing interpretation of electromagnetic interference as cardiac activity.

The above described implantable lead assembly allows for safe performance of the implantable lead assembly and pulse generator coupled thereto when exposed to electromagnetic interference. The magnetic jacket provides additional inductance with the inductance of the conductor within the implantable lead assembly. The added inductance of the magnetic jacket isolates the implantable lead assembly from electromagnetic interference, thereby decreasing transmission of interference to the pulse generator. In other words, the magnetic jacket prevents the pulse generator from falsely interpreting electromagnetic interference as cardiac activity.

Furthermore, the magnetic jacket is a compact and cost effective solution to the complicated and often large assemblies included in pulse generators. The magnetic jacket surrounds the conductor of the implantable lead assembly and therefore requires no volume within the pulse generator. The overall volume of the pulse generator is thus decreased. Because the magnetic jacket requires fewer parts and is easy to manufacture it decreases the cost and time to manufacture the implantable medical device.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is a block diagram of a system with a lead for use with a heart and constructed in accordance with one embodiment.

FIG. 2 is a first side view illustrating an implantable lead assembly constructed in accordance with one embodiment.

FIG. 3 is a cross sectional view illustrating an implantable lead assembly constructed in accordance with one embodiment.

FIG. 4 is a cross sectional view illustrating an implantable lead assembly constructed in accordance with another embodiment.

FIG. 5 is a cross sectional view illustrating an implantable lead assembly constructed in accordance with yet another embodiment.

FIG. 6 is a cross sectional view illustrating an implantable lead assembly constructed in accordance with still another embodiment.

FIG. 7 is a cross sectional view illustrating an implantable lead assembly constructed in accordance with an additional embodiment.

FIG. 8 is a cross sectional view illustrating an implantable lead assembly constructed in accordance with a further embodiment.

FIG. 9 is a block diagram illustrating one embodiment of a method of use for the implantable lead assembly.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

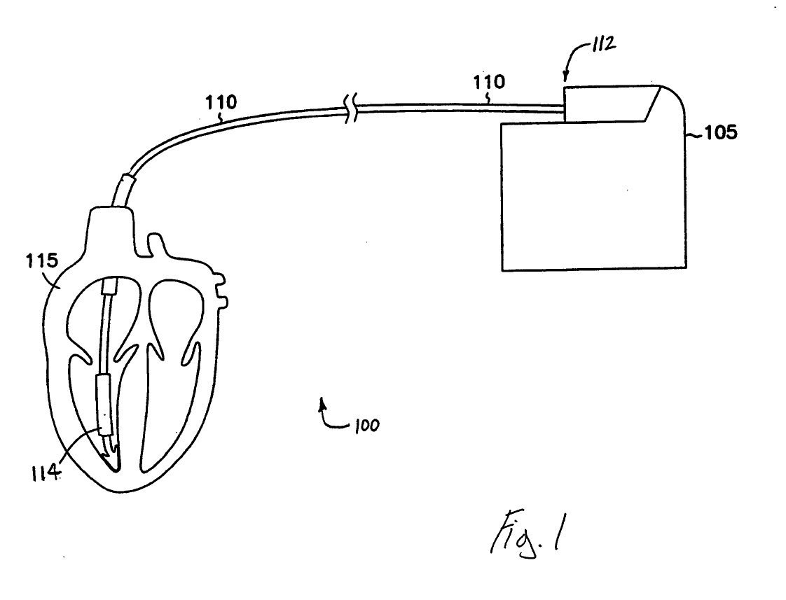

FIG. 1 is a block diagram of a system 100 for delivering and/or receiving electrical pulses or signals to stimulate and/or sense the heart. The system for delivering pulses 100 includes a pulse generator 105 and an implantable lead assembly 110. The pulse generator 105 includes a source of power as well as an electronic circuitry portion. The pulse generator 105 is a battery-powered device which generates a series of timed electrical discharges or pulses used to initiate depolarization of excitable cardiac tissue. The pulse generator 105 is generally implanted into a subcutaneous pocket made in the wall of the chest. Alternatively, the pulse generator 105 can be placed in a subcutaneous pocket made in the abdomen, or in other locations.

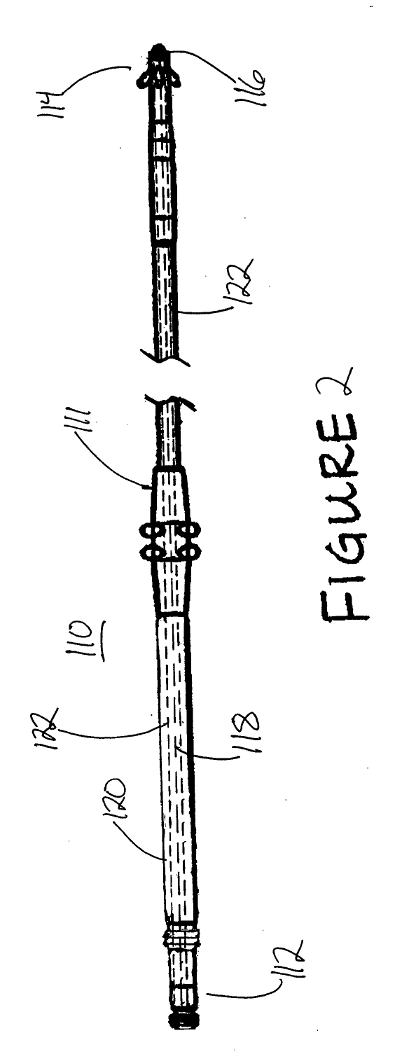

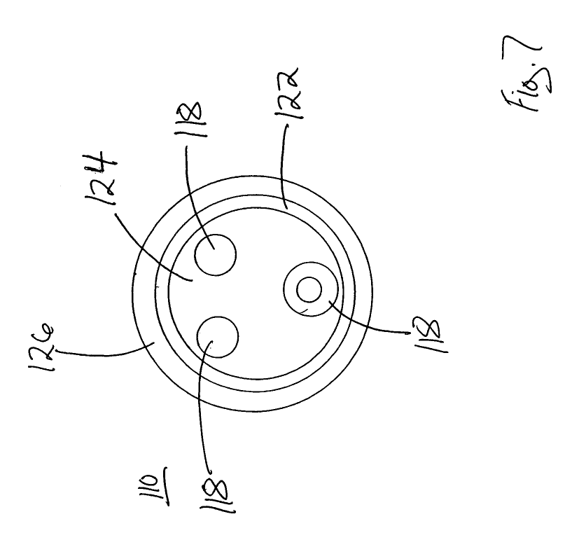

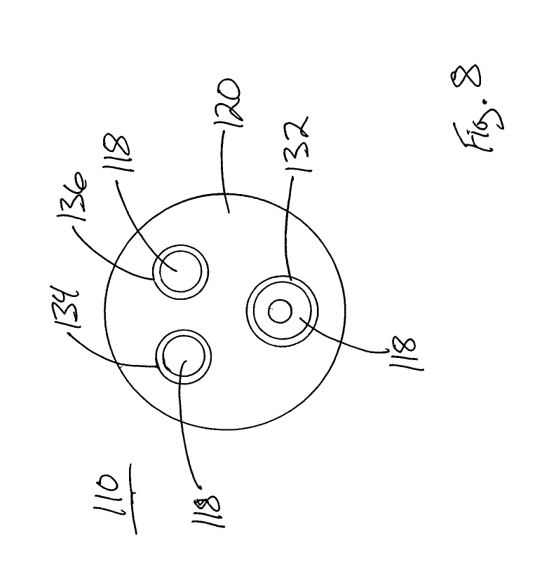

The implantable lead assembly 110, shown in more detail in FIG. 2, has a lead body 111 extending from a proximal end 112, where it is coupled with the pulse generator 105, and extending through an intermediate portion to a distal end 114, which, in one option, is coupled with a portion of a heart 115 in the implanted condition (one example is shown in FIG. 1). In another example, the lead body distal end 114 is disposed adjacent to the heart 115 and floats in a vein, in the implanted condition. In yet another example, the lead body distal end 114 is disposed within a chamber of the heart 115, and floats within the chamber. The distal end 114 of the implantable lead assembly 110 includes at least one electrode 116 which electrically couples the implantable lead assembly 110 with the heart 115. In one option, the electrode 116 is coupled with the lead body 111. The electrode 116, in one option, can be either a unipolar or multipolar type electrode. In one option, multiple electrodes are provided. At least one electrical conductor 118, as shown in phantom lines in FIG. 2, is disposed within the implantable lead assembly 110 and electrically couples the electrode 116 with the proximal end 112 of the implantable lead assembly 110. The electrical conductor 118 carries electrical current and pulses between the pulse generator 105 and the electrode 116 located in the distal end 114 of the implantable lead assembly 110. In another option, multiple electrical conductors 118 are disposed within the implantable lead assembly 110, as shown in FIGS. 7 and 8.

The lead body 111, in one option, includes an insulating layer 120 formed of a biocompatible polymer suitable for implementation within the human body. The insulating layer 120 is made from a silicone rubber type polymer, in one option. In another option, the insulating layer 120 includes polyurethane. In yet another option, the insulating layer 120 includes polyethylene terephthalate (PTFE). In still another option, the insulating layer 120 includes ethylene-tetrafluoroethylene (ETFE), or polysiloxane urethane. The insulating layer 120 surrounds and insulates the electrical conductor 118.

As shown in phantom lines in FIG. 2, one example of a magnetic jacket 122 is disposed within the insulating layer 120, in one option. The conductor 118 is disposed within the magnetic jacket 122, so the magnetic jacket 122 surrounds the conductor 118. In other words, the magnetic jacket 122 defines a perimeter around the conductor 118. The magnetic jacket 122 is aligned with a longitudinal axis defined by the conductor 118. In one option, the magnetic jacket 122 is electrically isolated from the electrode 116 and conductor 118 by the insulating layer 120. In another option, the insulating layer 120 also surrounds the magnetic jacket 122. The conductor 118 has a first inductance value. The magnetic jacket 122 has a second inductance value. In one option, the second inductance value is equivalent to the conductor 118 first inductance value. In another option, the second inductance value differs from the first inductance value.

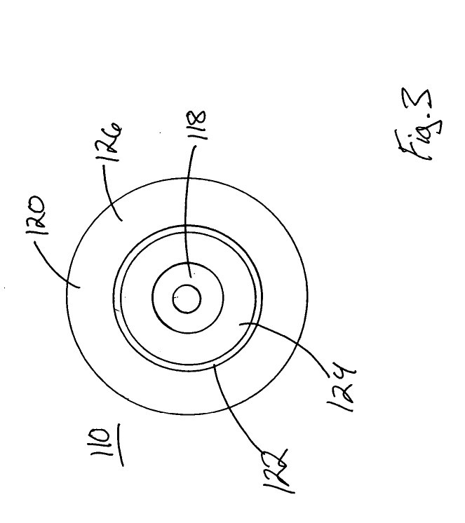

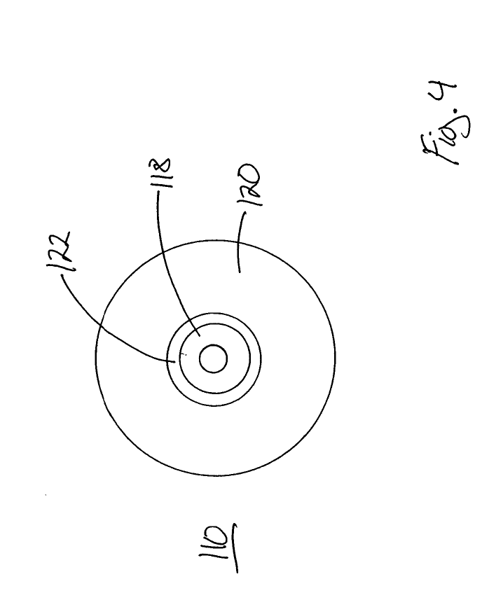

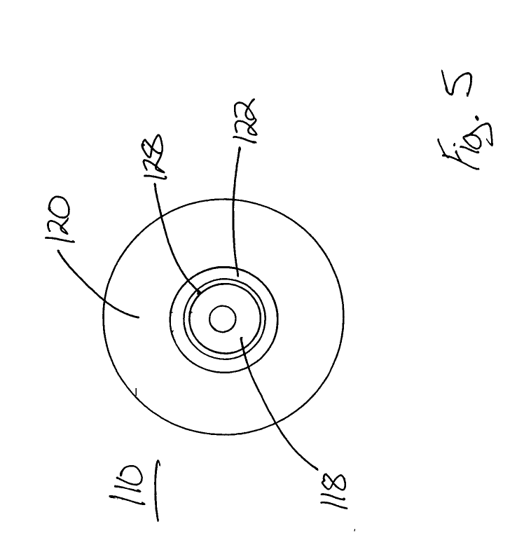

As shown in FIG. 3, in one option, the insulating layer 120 includes a first insulating portion 124 interposed between the conductor 118 and the magnetic jacket 122, and a second insulating portion 126 that surrounds the magnetic jacket 122. In other words, the magnetic jacket 122 is interposed between the first insulating portion 124 and second insulating portion 126. In another option, shown in FIG. 4, the magnetic jacket 122 surrounds the conductor 118 and is adjacent thereto. In yet another option, as shown in FIG. 5, the conductor 118 includes an insulating layer 128 of ETFE, PTFE, or other insulating material, which comprises the outer surface of conductor 118. The insulating layer 128 isolates magnetic jacket 122 from electrical communication with the conductor 118. Where the magnetic jacket 122 is nonconductive the insulating layer 128 is unnecessary, and the magnetic jacket may contact the conductor 118. Because the magnetic jacket 122 surrounds the conductor 118, the magnetic jacket substantially defines a perimeter around the conductor, as described above. In another option, the magnetic jacket 122 substantially surrounds the conductor 118 so as to define a broken perimeter around the conductor. In other words, the magnetic jacket 122 is a series of discrete elements disposed around the conductor 118, thus defining a non-continuous perimeter that substantially surrounds the conductor.

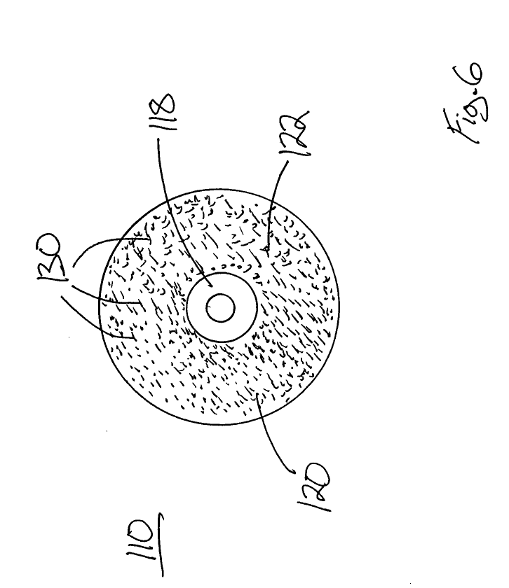

Referring now to FIG. 6, in one example, the magnetic jacket 122 and insulating layer 120 are coextensive in one or more parameters, for example, an outer diameter, inner diameter, or outer and inner diameters. In one option, the magnetic jacket 122 includes magnetic particles 130 (described below). These particles are disposed within interstitial spaces of the insulating layer 120. The magnetic particles 130 form the magnetic jacket 122 within the insulating layer 120. In one example, the magnetic particles 130 are mixed with a molten solution including the material used to form the insulating layer 120. The solution including the magnetic particles 130 is extruded around the conductor 118 to form the insulating layer 120 and coextensive magnetic jacket 122. In yet another option, the magnetic particles 130 are partially disposed within insulating layer 120. For example, a first portion of the insulating layer 120 is formed around the conductor 118 so as to surround the conductor. Magnetic particles 130 are included in the first portion of the insulating layer 120. A second portion of insulating layer 120 is formed around the first portion, and the second portion does not include magnetic particles 130. The magnetic jacket 122 is thereby disposed substantially adjacent to the conductor 118.

As shown in FIGS. 7 and 8, in another option, the implantable lead assembly 110 includes multiple conductors 118. As particularly shown in FIG. 7, in one option, the conductors 118 are disposed within the first insulating portion 124 so the first insulating portion surrounds the conductors. The first insulating portion 124 is disposed within a second insulating portion 126, in another option. The second insulating portion surrounds the first insulating portion 124 and conductors 118, in yet another option. In a further option, a magnetic jacket 122 is interposed between the first insulating portion 124 and second insulating portion 126. The magnetic jacket 122 surrounds the conductors 118, in still another option. The magnetic jacket 122 is formed from a substrate and magnetic particles disposed therein, in one option. Optionally, the magnetic jacket 122 is formed in another manner as described below.

Referring now to FIG. 8, in another example, the implantable lead assembly 110 includes multiple magnetic jackets 132, 134, 136 and corresponding multiple conductors 118. The conductors 118 and magnetic jackets 132, 134, 136 are disposed within the insulating layer 120, in one option. In another option, the insulating layer 120 surrounds the magnetic jackets 132, 134, 136 and conductors 118. In yet another option, the magnetic jackets 132, 134, 136 are nonconductive and disposed adjacent to the conductors 118 (See FIG. 8). The magnetic jackets 132, 134, 136 surround the conductors 118, in another option. In still another option, the conductors 118 include a thin insulating layer of ETFE, PTFE, or other suitable material comprising an outer surface of the conductors. The thin insulating layer electrically isolates the conductors 118 from the magnetic jackets 132, 134, 136. The magnetic jackets 132, 134, 136 are conductive, in another option, and disposed around the insulated conductors 118 so as to surround the conductors. In yet another option, each magnetic jacket 132, 134, 136 is offset from the respective conductor 118. In this option, the magnetic jackets 132, 134, 136 are disposed within the insulating layer 120, but still surround the respective conductors 118. Optionally, the magnetic jacket 122 includes magnetic particles 130, these particles are disposed within interstitial spaces of the insulating layer 120, as described above. The magnetic particles 130 thus form the magnetic jacket 122 within the insulating layer 120, in one option. In a further option, the magnetic jacket 122 is formed in another manner as described below.

In one option, the magnetic jacket 122 is formed from a thin coating of magnetic material, for example ferrite, applied to an insulating layer so as to surround the insulating layer and the conductor 118 disposed therein. The coating of magnetic material, in another option, is applied by sputtering or vapor deposition. In yet another option, the magnetic jacket 122 is a polymer substrate having magnetic particles. In still another option, the magnetic jacket 122 includes other substrate materials, for example, a shape memory alloy which contains magnetic particles. The magnetic jacket 122 is conductive, in another option. The magnetic jacket 122 is non-conductive, in still another option. In one example, where the magnetic jacket 122 is non-conductive, the magnetic jacket is applied directly to the conductor 118 so as to surround the conductor. In a further option, the implantable lead assembly 110 is created with alternating layers of polymer, where magnetic particle fillers are added to the polymer at various layers to create the magnetic jacket 122. Preferably, magnetic particles, particles of ferrite for example, are used in the magnetic jacket 122, though other particles with magnetic properties would suffice. In another option, the magnetic particles are interstitial particles disposed within interstitial spaces of the magnetic jacket 122. In yet another option, the magnetic particles are disposed within interstitial spaces of the insulating layer 120.

Referring again to FIG. 1, in operation the implantable lead assembly 110 is coupled to the pulse generator 105 and this system 100 is generally implanted within the chest. The distal end 114 of the implantable lead assembly 110 is coupled to the heart 115 or disposed adjacent thereto. The conductor 118 (FIG. 2) is electrically coupled to the heart by the electrode 116 (FIG. 2). The implantable lead assembly 110 is exposed to electromagnetic interference, for example, interference created by cellular phones, theft deterrent systems or microwave ovens, when the patient is in close proximity to these sources. The first inductance value of the conductor 118 and the second inductance value of the magnetic jacket 122 (FIGS. 2-7) combine for a third inductance value, in one option. The third inductance value is greater than the first inductance value of the conductor 118. In other words, the inductance of the conductor 118 is supplemented with the inductance of the magnetic jacket 122. In yet another option, the inductance of multiple conductors 118 is supplemented with the inductance of the magnetic jacket 122. The increased inductance of the implantable lead assembly 110, due to the added inductance from magnetic jacket 122, limits the amount of electromagnetic interference that is otherwise transmitted along the implantable lead assembly to the pulse generator 105. The implantable lead assembly 110 and pulse generator 105 are thereby isolated from electromagnetic interference by the magnetic jacket 122 that surrounds the conductor 118. The magnetic jacket 122 thus reduces the susceptibility of the pulse generator 105 to falsely interpreting electromagnetic interference as cardiac activity. In other words, the antenna effect of the implantable lead assembly 110 is reduced by the magnetic jacket 122, thereby permitting safe operation of the implantable medical device without electromagnetic interference.

Additionally, where there are multiple conductors 118 and a single magnetic jacket 122 or multiple magnetic jackets 132, 134, 136, the effect is similar to that previously described. In one option, the inductance value of the magnetic jacket 122 (FIG. 7) combines with the inductance values of the multiple conductors 118 to increase the inductance of the implantable lead assembly 110. In another option, the inductance values of the multiple magnetic jackets 132, 134, 136 (FIG. 8) combine with the inductance values of the conductors 118 to increase the implantable lead assembly 110 inductance. The implantable lead assembly 110 and pulse generator 105 are isolated from electromagnetic interference. Additionally, the pulse generator 105 has reduced susceptibility to falsely interpreting electromagnetic interference as cardiac activity. The antenna effect of the implantable lead assembly 110 is reduced, allowing safe operation of the implantable medical device.

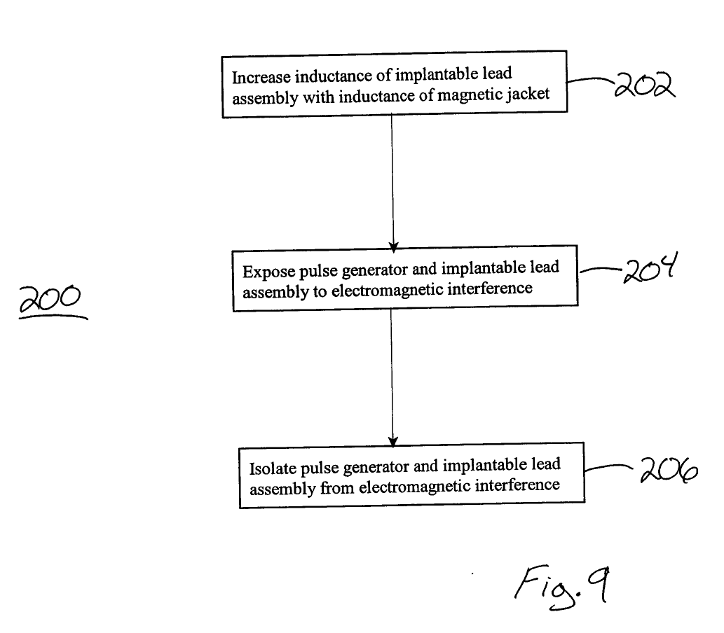

In another embodiment shown in FIG. 9, a method 200 comprises increasing the inductance of an implantable lead assembly with the inductance of a magnetic jacket, as principally shown in block 202. The implantable lead assembly includes a lead body extending from a proximal end to a distal end. The lead body includes an insulating layer and a conductor disposed therein. An electrode is coupled to the lead body and is in electrical communication with the conductor. The insulating layer surrounds the conductor and the magnetic jacket is disposed within the insulating layer. The magnetic jacket surrounds the conductor. As shown in block 204, the method further includes, exposing a pulse generator and the implantable lead assembly coupled thereto to electromagnetic interference. The method additionally includes isolating the pulse generator and the implantable lead assembly from electromagnetic interference, as shown in block 206.

Several options for the method follow. In one option, increasing the inductance of the implantable lead assembly (block 202) further includes adding the inductance of the magnetic jacket with an inductance of the conductor. In another option, isolating the pulse generator and the implantable lead assembly from electromagnetic interference (block 206) further includes decreasing the antenna efficiency of the implantable lead assembly. In other words, the antenna effect of the implantable lead assembly is decreased. In yet another option, isolating the pulse generator and the implantable lead assembly further includes decreasing interpretation of electromagnetic interference as cardiac activity. The method 200 further includes sensing cardiac activity with the electrode and conductor.

The above described design for an implantable lead assembly allows for improved performance of the implantable lead assembly and pulse generator coupled thereto when exposed to electromagnetic interference. The magnetic jacket surrounds the conductor and provides additional inductance to the conductor within the implantable lead assembly. The added inductance of the magnetic jacket isolates the implantable lead assembly from electromagnetic interference, thereby decreasing transmission of the interference to the pulse generator. In other words, the magnetic jacket assists in preventing the pulse generator from falsely interpreting electromagnetic interference as cardiac activity.

Furthermore, the magnetic jacket is a compact and cost effective solution to the complicated and often large assemblies included in pulse generators. The magnetic jacket surrounds the conductor of the implantable lead assembly and therefore requires no volume within the pulse generator. The overall volume of the pulse generator is thus decreased. Because the magnetic jacket requires fewer parts and is easy to manufacture it decreases the cost and time to manufacture the implantable medical device. The implantable lead assembly and the methods described above may also be used in other implantable medical lead applications beyond cardiac pacemakers, for example, spinal cord and brain stimulation implants, urinary implants, cochlear implants, or in other devices utilizing stimulation and/or sense electrodes.

It is to be understood that the above description is intended to be illustrative, and not restrictive. Many other embodiments will be apparent to those of skill in the art upon reading and understanding the above description. It should be noted that embodiments discussed in different portions of the description or referred to in different drawings can be combined to form additional embodiments of the present application. The scope of the invention should, therefore, be determined with reference to the appended claims, along with the full scope of equivalents to which such claims are entitled.

1. An implantable lead assembly comprising:
a lead body extending from a proximal end to a distal end having an intermediate portion therebetween, wherein the lead body includes an insulating layer;
a conductor disposed within the insulating layer, wherein the insulating layer surrounds the conductor;
an electrode coupled to the lead body, wherein the electrode is in electrical communication with the conductor; and
at least one magnetic jacket disposed within the insulating layer, wherein the at least one magnetic jacket surrounds the conductor.

2. The implantable lead assembly of claim 1, wherein the at least one magnetic jacket is disposed adjacent to the conductor.

3. The implantable lead assembly of claim 2, wherein the at least one magnetic jacket is electrically nonconductive.

4. The implantable lead assembly of claim 2, wherein the conductor includes a second insulating layer comprising the outer surface of the conductor, the second insulating layer electrically isolates the conductor from the at least one magnetic jacket.

5. The implantable lead assembly of claim 1, wherein the insulating layer includes a first portion and a second portion, the first portion surrounds the conductor, the second portion surrounds the first portion, and the at least one magnetic jacket is interposed between the first portion and the second portion.

6. The implantable lead assembly of claim 1, wherein the at least one magnetic jacket includes a substrate and magnetic particles, and the magnetic particles are disposed within the substrate.

7. The implantable lead assembly of claim 1, wherein the at least one magnetic jacket is coextensive with the insulating layer, the at least one magnetic jacket includes interstitial magnetic particles, and the interstitial magnetic particles are disposed within the insulating layer.

8. The implantable lead assembly of claim 7, wherein the interstitial magnetic particles are disposed throughout the insulating layer.

9. The implantable lead assembly of claim 1, wherein a second conductor is disposed within the insulating layer, and the insulating layer surrounds the second conductor.

10. The implantable lead assembly of claim 9, wherein the at least one magnetic jacket surrounds the second conductor.

11. The implantable lead assembly of claim 9, wherein a second magnetic jacket is disposed within the insulating layer, and the second magnetic jacket surrounds the second conductor.

12. An implantable lead assembly comprising:
a lead body extending from a proximal end to a distal end having an intermediate portion therebetween, wherein the lead body includes an insulating layer;
a conductor disposed within the insulating layer, wherein the insulating layer surrounds the conductor, the conductor has a first inductance value;
at least one magnetic jacket disposed within the insulating layer, wherein the at least one magnetic jacket has a second inductance value; and
an electrode coupled to the lead body, wherein the electrode is in electrical communication with the conductor.

13. The implantable lead assembly of claim 12, wherein the at least one magnetic jacket surrounds the conductor.

14. The implantable lead assembly of claim 12, wherein the conductor and at least one magnetic jacket have a combined third inductance value, and the third inductance value is greater than the first inductance value.

15. The implantable lead assembly of claim 12, wherein the at least one magnetic jacket includes a substrate and magnetic particles, and the magnetic particles are disposed within the substrate.

16. An implantable lead assembly comprising:
a lead body extending from a proximal end to a distal end having an intermediate portion therebetween, wherein the lead body includes an insulating layer;
a conductor disposed within the insulating layer, wherein the insulating layer surrounds the conductor;
an electrode coupled to the lead body, wherein the electrode is in electrical communication with the conductor; and
means for isolating the conductor from electromagnetic interference.

17. The implantable lead assembly of claim 16, wherein the means for isolating the conductor from electromagnetic interference includes a magnetic jacket disposed within the insulating layer, the magnetic jacket surrounds the conductor.

18. The implantable lead assembly of claim 17, wherein the magnetic jacket is electrically isolated from the conductor by a second insulating layer, the second insulating layer surrounds the conductor.

19. The implantable lead assembly of claim 17, wherein the magnetic jacket includes a substrate and magnetic particles, and the magnetic particles are disposed within the substrate.

20. A method comprising:
increasing the inductance of an implantable lead assembly with the inductance of a magnetic jacket, the implantable lead assembly including a lead body extending from a proximal end to a distal end, the lead body includes an insulating layer and a conductor disposed within the insulating layer, an electrode is coupled to the lead body and in electrical communication with the conductor, the insulating layer surrounds the conductor, and the magnetic jacket is disposed within the insulating layer and the magnetic jacket surrounds the conductor;
exposing a pulse generator and the implantable lead assembly coupled thereto to electromagnetic interference; and
isolating the pulse generator and the implantable lead assembly from electromagnetic interference.

21. The method of claim 20, wherein increasing the inductance of the implantable lead assembly further includes adding the inductance of the magnetic jacket with an inductance of the conductor.

22. The method of claim 20, wherein isolating the pulse generator and the implantable lead assembly further includes decreasing an antenna efficiency of the implantable lead assembly.

23. The method of claim 20, wherein isolating the pulse generator and the implantable lead assembly further includes decreasing interpretation of electromagnetic interference as cardiac activity.
